(19) United States
(12) Patent Application Publication
(10) Pub. No.: US 2010/0121419 A1
(43) Pub. Date: May 13, 2010

(54) CONTROL OF LIGHT SOURCES FOR LIGHT THERAPIES

(76) Inventor: Ryan Douglas, Stillwater, MN (US)

Correspondence Address: DARDI & HERBERT, PLLC, 220 S. 6TH ST., SUITE 2000, U.S. BANK PLAZA, MINNEAPOLIS, MN, 55402, US

(21) Appl. No.: 12/617,916

(22) Filed: Nov. 13, 2009

Related U.S. Application Data
(60) Provisional application No. 61/114,366, filed on Nov. 13, 2008.

(51) Int. Cl. A61N 5/06

(52) U.S. Cl. 607/90; 607/88

(57) ABSTRACT

Light therapy systems that track their own usage and provide for control of light output. LEDs can degrade over time in a way that may not be apparent to a user but impacts therapy. A light therapy system activates a light source and applies therapeutic light to a tissue of a patient during a session. Usage data is stored over a plurality of the sessions and is tested against a predetermined value to determine when further degradation of the light source is unacceptable.

### CROSS-REFERENCE TO RELATED APPLICATIONS

This application claims priority to U.S. Ser. No. 61/114,366 filed Nov. 13, 2008, which is hereby incorporated by reference herein.

### TECHNICAL FIELD

The technical field relates to phototherapy, especially the use of light emitting diodes to treat a tissue for therapeutic purposes.

### BACKGROUND

Phototherapy relates to administering doses of light of a certain wavelength or spectrum of wavelengths. The light has an intensity, with the product of intensity and time resulting in a dose. A measure of intensity is Watts/cm². The dose is typically applied per unit area; a measure of dosage is J/cm², i.e., the product of Watts/cm² and time. A dose may be administered in a session and a dosage regimen performed by administering a dose per time interval, e.g., one session per day.

### SUMMARY

Devices are described for applying therapeutic light that is at the correct wavelength, for the correct amount of time, at the necessary interval (e.g., doses per day), and at the correct intensity. LEDs tend to fade in intensity over time as they degrade, in contrast to conventional light bulbs that tend to fail suddenly. Moreover, degradation of LEDs can result in a shift of the wavelength emitted from the LED. These problems are exacerbated when LEDs are packed in an array and enclosed in a housing because the LEDs become exposed to heat that is much greater than the test conditions that are used for testing their lifetime at the original equipment manufacturer (OEM). In other words, standard testing conditions do not properly predict the actual time that a LED will provide its rated output and wavelengths in a phototherapy device.

The number, type, arrangement, and pattern of use of the LEDs in a phototherapy device all affect the heat accumulation and life of the LEDs. Further, the therapeutic application also has an indirect affect on LED lifetime. For instance, a dosage regimen of five minutes in a session can generate heat and conditions distinct from a regiment of ten minutes at a session.

### BRIEF DESCRIPTION OF THE FIGURES

FIG. 1 is a perspective view of a hand-held system for providing therapeutic light;

FIG. 2 is a perspective view of the system of FIG. 1 in a charging position;

FIG. 3 is an enlarged perspective view of the reversible attachment of the heads of FIG. 1 to the body to form a unit;

FIG. 4 is an enlarged perspective view of a head of FIG. 1;

FIG. 5 is an enlarged view of an embodiment of a control panel of FIG. 1;

FIG. 6 is a perspective view of a hand held phototherapy unit in use on a facial tissue;

FIG. 7 is a flow chart of an embodiment of a light usage and control system;

FIG. 8 is a perspective view of a system for providing hand-held therapy, with computer readable media for each of a plurality of heads;

FIG. 9 is a perspective view of a unitary light phototherapy unit.

### DETAILED DESCRIPTION

Phototherapy devices are provided herein that track their own usage and permanently shut themselves down when the device performance begins to fail. In one embodiment, a system has a plurality of heads that reversibly connect to a single body. The heads have LEDs or other light sources that provide phototherapeutic light. The usage of each head is tracked. When usage passes a predetermined threshold value (the predetermined test value), the head will no longer be activatable.

In one embodiment the heads have a computer readable medium that stores usage information. The medium is read by a processor in the system and updated. When the updated value passes the test value, the processor, or other processors in the system control the head so that it will not start. Alternatively or additionally, the system may display a signal to a user indicating a status of the head life, e.g., good, nearing expiration, or expired.

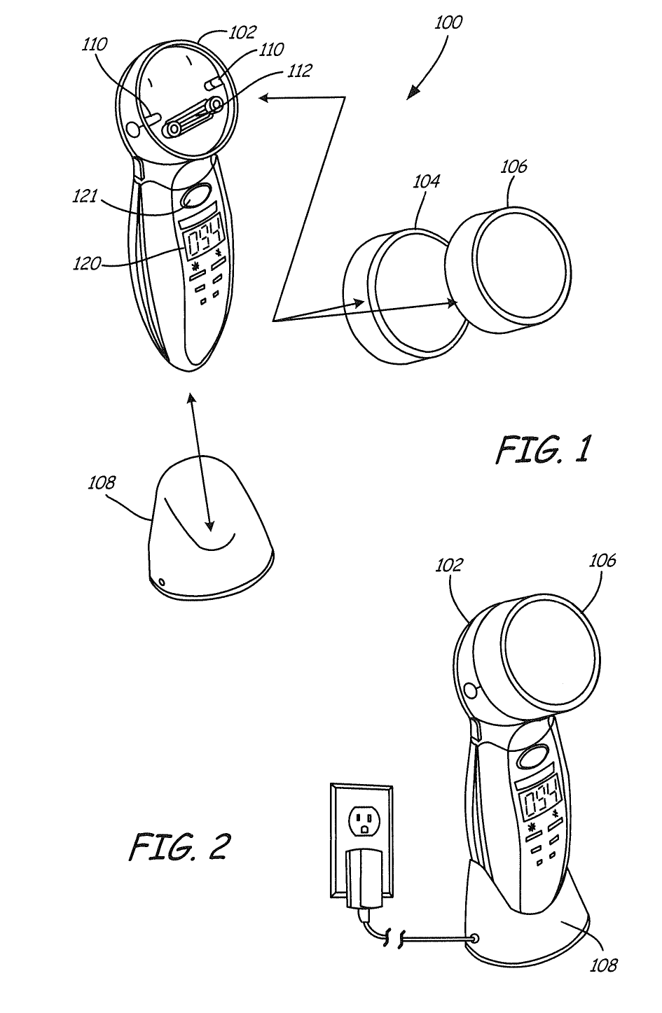

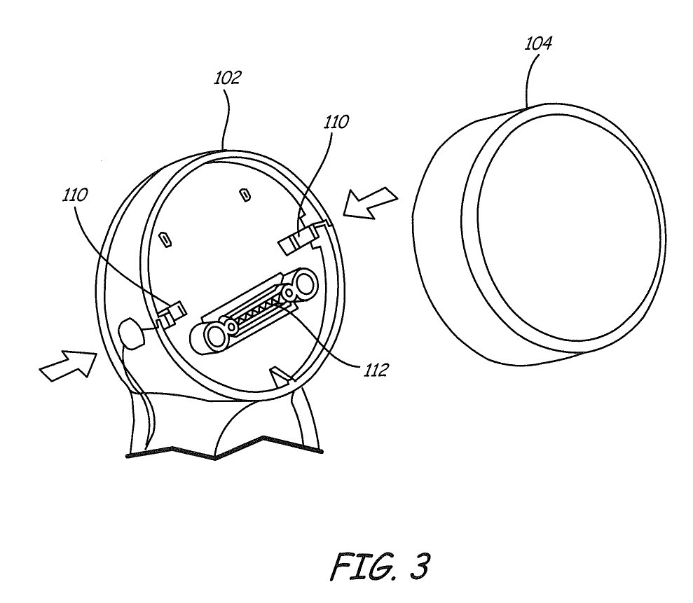

FIG. 1 depicts an embodiment of a hand-held user-activatable phototherapy system that includes a plurality of light-emitting heads. System 100 has body 102, heads 104, 106, and base 108 that cooperate as shown at FIG. 2. Heads 104, 106 are reversibly connectable to base 102 by fasteners 110, see FIG. 3. Electrical connector 112 provides for passage of electrical power from body 102 to a head that is connected.

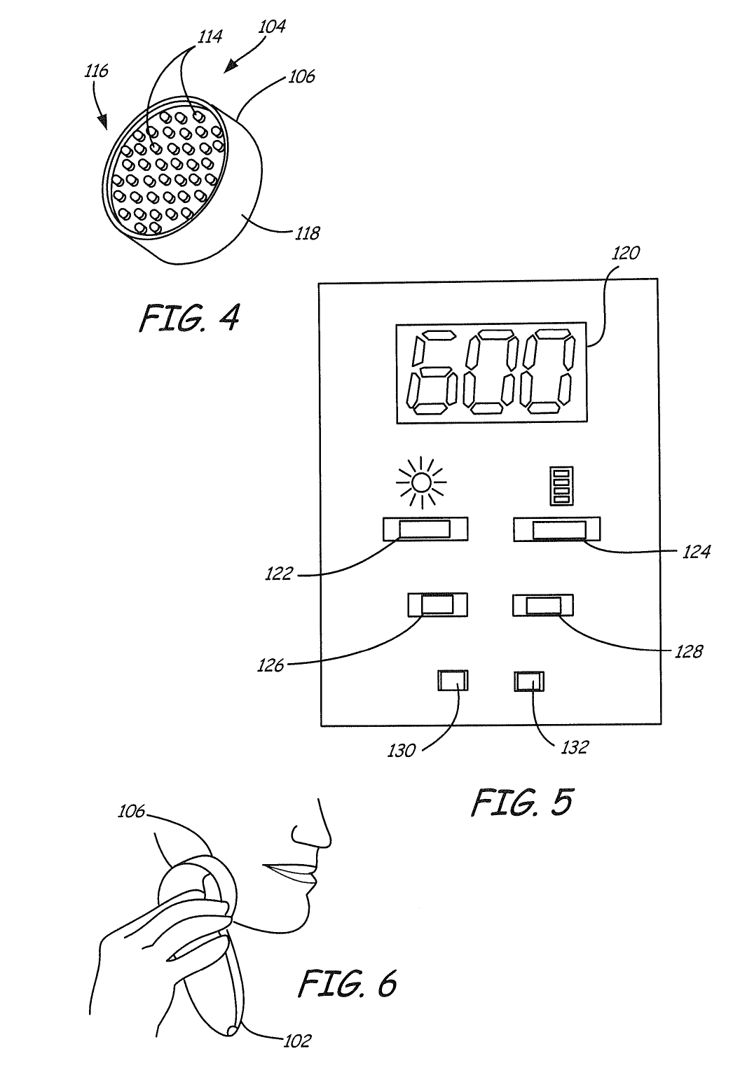

Heads 104, 106 have a plurality of light emitting diodes (LEDs) 114 arranged in an array 116, as at FIG. 4. Alternative light sources may be used, or LEDs may be used in combination with other light sources. For instance, one or more laser diodes may be added to, or substituted for, one or more LEDs, e.g., a red LED may be a red laser diode, or a blue laser diode may be included. A variety of array arrangements may be used, with the number of light sources, their intensity, and/or packing being varied.

Slot 118 receives reversible fastener 110. Fasteners may be used as helpful, e.g., a biased tab that cooperates with a slot, a lip that provides a friction fit, a threaded connection, a tab-and-slot for twist on-and-off connection. Heads are generally depicted as being circular but may have other shapes, e.g., oval, square, concave, concave-oval, convex. Body 102 passes electrical power from a power source, e.g., a wall outlet or has a battery for providing power. Body 102 has display 120, on/off control 121, and indicators 122, 124, 126, 128, 130, 132, see FIGS. 1 and 5.

A user activates the LEDs in the head with the on/off control 121 and directs the light from the LEDs onto a tissue by holding the head by the tissue, as at FIG. 6. Body 102 display 120 provides information to the user, e.g., amount of time left in a session, the session time, remaining uses or life in the head, or intended therapy, for instance, acne, wrinkles, baldness. Other indicators provide information, e.g, battery life, light longevity. Audio tones or speech may be provided by the unit to give indicia and/or instructions.

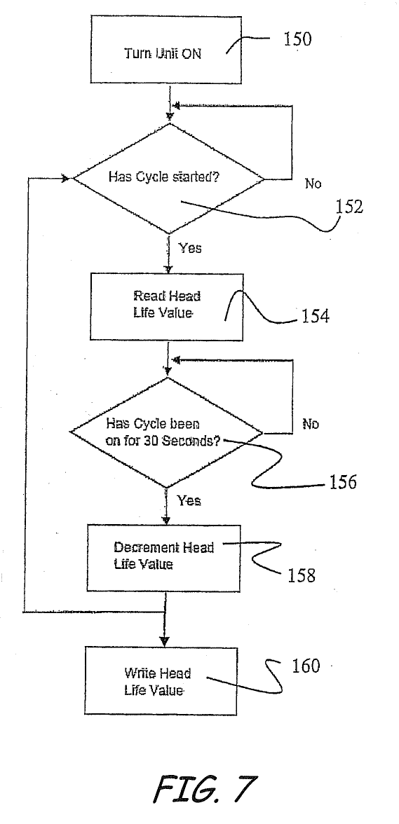

Some embodiments provide a tracking system to track the usage of a head and control head activation or display head usage information. FIG. 7 is a flow chart of such an embodiment. User turns on the unit, 150, a processor checks to see if a light source activation cycle has been started, 152. If NO, then the processor queries the system again. If YES, the processor reads, 154, the head life (usage) value from a computer-readable medium located in the system (e.g., head, body, base, desktop computer, console, remote processor). The processor tests to determine if the cycle has been “on” for 30 seconds, 156. If NO, then the processor again queries the system for a determination the cycle has been on, with the queries being stopped in the case that the unit is turned off. If YES, then the processor decrements the head life value, 158. The new value is written, 160 to the computer-readable usage value location.

Alternative embodiments are similar to that of FIG. 6, with the usage value being a computer-readable medium located in the head, the body, base, or other location. The usage value is tested and the result of the test provides for an instruction. The instruction may include a command to prevent activation of lights in the head or provide a certain display or other signal, e.g., audio tone. In some cases no instruction is issued if the test has a certain result. Or a negative result could cause a first instruction and a positive result could result in a second instruction.

The processor or processors for performing the tests may be located in the head, the body, base, or other location. The various tests may be performed by one or more of the processors located in one or more of the locations. The term processor is broad and includes devices such as a general purpose computer, a user-programmable chip, and a chip programmable prior to assembly of the device. Accordingly a processor is a term that includes an integrated circuit (also known as IC, microcircuit, microchip, silicon chip, or chip), a miniaturized electronic circuit (semiconductor devices and/or passive components). And a computer-readable medium is readable by the processor, e.g., flash storage media, permanent or temporary memory, a registry, random-access memory (RAM), dynamic RAM (DRAM), Z-RAM, TTRAM, A-RAM, volatile memory, non-volatile memory, read-only memory (see ROM), magnetic computer storage devices, and optical memory.

The processor, or one of a plurality of processors in a device or system of devices, may be a counter. A counter may be a software counter controlled by a microcontroller, e.g., on the main board. Alternatively, an electronic hardware counter may be provided to perform the same function. There are electronic components that are designed to be counters and these could be configured to work as count-down counters, or incrementing (count-up) counters. Alternatively, other electronic components, which are not microcontrollers, may be programmed to act as counters. Some of these devices are called programmable logic devices (PLD) and field programmable gate arrays (FPGA). There are many different variations of these in the market place having the requisite capabilities and functionality.

For instance, a hardware counter could work as follows: A preset value can be loaded into a hardware counter upon start-up. After a trigger is provided that indicates that a treatment cycle has been operating for predetermined threshold time (e.g., 30 seconds) this trigger will cause the countdown counter to decrement by one. Once the counter has reached zero, an output will activate, which can be used to halt any more use of the device.

Counters may be included in the device such that they are in electronic communication with the device as needed to capture the appropriate parameter. In the case of systems with a plurality of detachable heads that each have one or more LEDs, a counter may be located in the head. The counter communicates with another portion of the device equipped with circuitry or microprocessors to query the counter and take action, e.g., provide a display to a user that is audible and/or visual, indicate the time or cycles of life remaining in the head, indicate a need for replacement, or prevent activation of the LEDs or the head itself. For instance, if the head attaches to a main body, the main body may have hardware or software logic to provide the intended action. Or if the head is in communication with a base unit, either directly or indirectly, by wire or wireless, the base unit may perform the processing role. Alternatively, in the case of systems with a plurality of detachable heads that each have one or more LEDs, a counter may be located in the unit the head attaches to. Or the counter may be in a base unit that received the unit the head attaches to. The counter may be incremented or decremented with each use or track predetermined parameters. Similarly, a processing logic device may be located in a head, base unit, or other member.

The usage value may take a variety of forms. One embodiment is an integer value that represents the number of sessions, e.g., the number of uses that exceed a threshold activation time such as the 30 seconds of FIG. 7. The usage value is incremented for each session. The value is tested against a predetermined test value and the head or other system component. The result of the test determines whether or not the head has exceeded a predetermined number of uses. Alternatively, a decrement-driven system may be used, with a predetermined number of uses being provided as the test value and being decremented upon each use. Another usage value embodiment is a time. The cumulative time that a head is activated is incrementally added to the usage value so that it reflects a cumulative duration of usage. Alternatively, the usage value represents a time and is decremented according to each usage. In some embodiments, the incrementing or decrementing is all-or-nothing, while other embodiments provide for varying session times that result in a plurality of updates to the value, e.g., each minute of a session causes a decrement, with a typical session being 5-10 minutes. The testing function may take a variety of forms. Accordingly, an outcome of a test may depend on whether the test value is >, ≧, =, ≦, <, ≠ or to a tested value.

A predetermined test value may take a variety of forms. Various parameters are set forth herein that may serve as a predetermined test value, e.g., total time, total uses, estimated degradation, degradation caused by driving power and/or temperature and/or LED materials composition. A test value may be a real number or an integer. For purposes of claiming the invention “a test value” refers to one or more test values. Accordingly, embodiments include a single test value, two test values, or any number of values, e.g., between 2-10 test values; artisans will immediately appreciate that all the ranges and values within the explicitly stated ranges are contemplated.

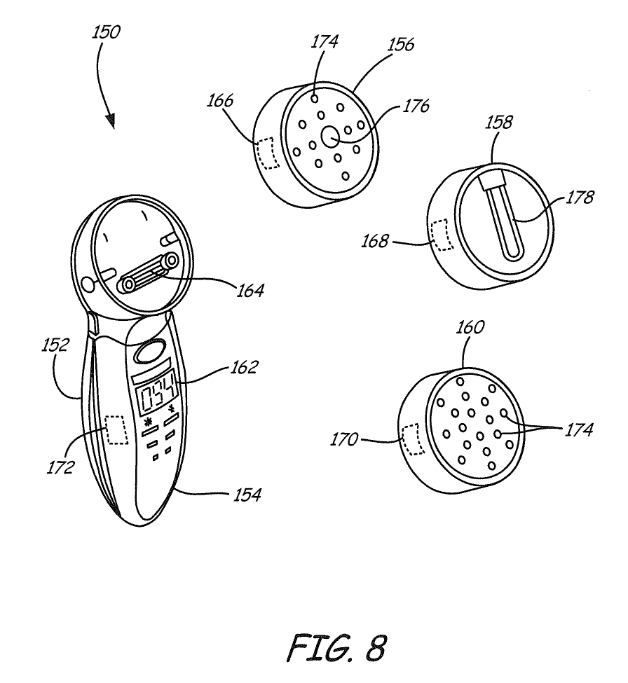

One embodiment places the usage value and the test value in the head, e.g., by placing them in a computer-readable memory in the head. A processor (in the head, the body, or other location) reads the usage value and the test value and performs a test of the usage value against the test value. The test controls an outcome. For instance, the processor may direct the body to pass no power to the head. The processor then writes a new usage value to the usage value location, e.g., increments or decrements the same. Another embodiment places the usage value in the head and the test value in another location, e.g., the body or the base, see FIG. 8. The processor performs the test, controls the outcome, and writes the update to the usage value. Similarly, the usage value and the test value may both be outside the head, e.g., in the body or a base, with the head having a stored head-identifier. The processor recognizes the identifier and performs its test, control, and updating functions accordingly.

The control of the light activation may be performed in a variety of ways. For instance, a switch may be provided in the head or in the body. One embodiment provides a deactivation switch in the head. A test result indicating that there is to be no further usage of the head causes the deactivation switch to be moved so that the head is deactivated and can no longer provide power to the light sources. Alternatively, a prevent-switch may be provided in the body that prevents passage or electrical power to the head when a test result indicates the head's life is over. Or, a test result that allows activation may be required, with the default result being no activation of the head. The term switch includes mechanical, electromechanical, and electronic switches.

Testing of head usage can result in a audio or visual display. Sounds or symbols may be used to indicate the amount of cumulative usage or alternatively, the amount of lifetime left in the head. Examples of a visual display are words (e.g., high, medium, low), lights (green, red, blue), bars (short, medium long), percentages (100% to 0%). The device may track the usage of the LED, including, for instance one or more of runtime (duration of light source activation, i.e., how long the light is “on”), actual or estimated operating temperatures, and light source drive current, and be dynamically adjusted to account for the remainder of useful life for the LEDs relative to the use patterns. For instance, the system may permanently disable the use of LEDs that have surpassed their calculated useful therapeutic life, ensuring the end user does not continue to use reduced output LEDs unknowingly receiving a reduced therapeutic dose that could negatively impact the intended and tested treatment effects of the device.

FIG. 8 depicts a system 150 for providing light therapy with a hand-held unit 152. Body 154 is reversibly connectable to a heads 156, 158, 160, each of which are directed to a different therapeutic application. Body 154 has a display 162 and electrical connection 164 for reversible connection to each head. Heads have computer readable media 166, 168, 170 that receive updates from processor 172. The heads have various light sources including LEDs 174, laser diode 176, and light bulb 178. A user connects a head to body 154 to form hand-held unit 152, activates the unit, and places the head against a tissue that receives light from the light source. Processor 172 determines that the source is activated and reads usage information from the connected head, tests the information against a predetermined value, and issues an instruction as a result of the test. The instruction can provide that the head is not activated if the usage fails a comparison based on a predetermined value (e.g., if the usage meets the test value, the head will not activate). Alternatively, it can issue an instruction to display a status of the head or one or more light sources. The processor updates the usage information to the computer readable medium on the connected head. Each head thus carries its own usage history and the system tracks its own usage history.

To determine of light source degradation exceeds a predetermined test value refers to programming a processor to test for unacceptable or not-desired degradation. This terminology thus includes all the various permutations for programming a test of a variable against a test value. By way of example, a programmer that wishes a head to shut itself off after 1000 hours of run time can program the processor to allow light activation only if the run time is less than 1000, or allow activation unless the test shows exactly 1000 hours. Or the programmer could make the test value be 0 and decrement the run time variable. The programming can thus be set up to determine if the degradation exceeds a predetermined value regardless of whether the code calls for an equal-to or a less-than test.

Embodiments of the invention include these or other systems that have LEDs used for therapeutic applications and also track LED cycles, including for example (a) time of LED in operation, (b) number of cycles of LED, or use LED operation parameters of one or more of (i) LED time in operation, (ii) LED amperage in operation, or (iii) LED voltage in operation. Accordingly, for example, one embodiment tracks the product of LED time and amperage in operation or tracks these parameters separately and calculates said product. Another example is a counter that tracks the total time of an LED in operation. Another example is a counter that tracks the number of uses of an LED. One LED may be tracked, a plurality of LEDs may be tracked collectively or individually, or in groups (e.g., red LEDs and blue LEDs tracked separately) or an array of LEDs may be tracked. Accordingly, the total time that a detachable head that comprises one or more LEDs may be tracked using one or more of any of these parameters. For instance, the number of times a user initiates a cycle of treatment may be counted.

Light emitting diodes do not function like many other lighting sources by experiencing total operational failure when the light source life has been reached. The light output of a LED decreases over time to a point where the light output level is not sufficient for its intended application even though the LED may still illuminate. At this point, the light source is considered to be at the end of its useful life and should be replaced. In the case of use of light for therapy applications, the degradation and resulting reduction in dosage can have considerable image on the effectiveness of the therapy.

In fact, as little as a 5% deviation could impact the effectiveness of at least some therapies. By way of contrast, the Alliance for Solid-State Illumination System and Technologies, ASSIST defines LED life as the time that it takes the light output from a component or system to reach a level of sufficiency in comparison to its original/initial output, with the suggested limits of sufficiency defined as 50% of its initial output efficiency for decorative light sources, 70% of its initial output efficiency for general light sources, and 80% of its initial output efficiency for light sources where output is critical to its function (such as warning lights, required illumination levels or critical functioning components of a system that rely on light output for accurate operation).

Herein, however, LED use may be tracked and the device automatically becomes inoperative or notifies the user when a predetermined parameter indicates that output efficiency has dropped to or beyond a predetermined setting. Some of the predetermined parameters have already been described, e.g., number of use cycles or total time of use. The predetermined setting to shut-off or indicate a need for replacement may take place so that the loss of output efficiency is no more than 1%-40% of initial output efficiency; artisans will immediately appreciate that all the ranges and values within the explicitly stated ranges are contemplated, e.g., 2%, 5%, 9%, 10%, 15%, from 1% to 20%, 2% to 30%, 5% to 15%, or 5% to 10%.

Light emitting diodes (LEDs) are rated for their long life expectancies. The term LED does not include a laser diode for purposes of claiming an invention. These life estimate rating tests quoted by manufacturers (OEMs) are usually completed on a single component under specific controlled conditions which place the least amount of stress on the LEDs ability of functionality. The actual life expectancy and rate of degradation of an LED is dependant on factors such as the drive current, the ambient temperature, and the system's surrounding environment. The system's surrounding environment includes the number of LEDs in the system, the spacing between the LEDs in the system, and the types of system the LEDs are incorporated into (closed or open).

When considering LEDs for delivery of therapeutic light, it is important to note that the nature of the therapies and the devices that deliver the light often require tight groupings of LEDs that are driven to or beyond optimum output level to achieve efficient and effective treatments. This type of use produces additional heat, further impacting the LEDs ability to produce consistent dosing and therefore the unit's useful therapeutic life. Indeed, although white LEDs degrade faster than blue, overdriving white LEDs (milliamperes ranging from 20 to 50) has increased the degradation rate by 15% to 40% after 2000 hours.

The manufacture's lifetime rating test data may be used when programming or configuring a device, with the parameters in the specific device being adjusted to reflect actual test data, which can vary from batch to batch of products. Accordingly, some embodiments are that each device or lot of devices are to be customized to the LEDs that are used, as opposed to a generalized approach that may be less accurate. Accordingly embodiments are provided that include accounting for test data that includes one or more the following: the number of LEDs in the batch, the number of hours the batch was tested for, the ambient temperature that batch was controlled at, the drive current at which the LED batch was maintained at, and the LED failure rate for the test duration.

References, hereby incorporated herein by reference; in case of conflict, the specification controls: (1) Jennifer Taylor, “Industry alliance proposes standard definition for LED life” LEDs Magazine. 2005, see www for ledsmagazine.com. (2) Nadarajah Narendran, Jean Paul Freyssinier, Jennifer Taylor, “LED Luminaire Performance: Changing Traditions can set the right expectations” LEDs Magazine. 2007 see www for ledsmagazine.com/features/4/12/1. (3) Nadarajah Narendran, Jean Paul Freyssinier, Jennifer Taylor, “Common Questions”. (4) Nadarajah Narendran, Jean Paul Freyssinier, Jennifer Taylor, “The Impact of Temperature and Phosphor Concentration on the Refractive Index of LEDs Encapsulants” see www for lrc.rpi.edu/programs/solidstate/cr impact.asp (5) Melanie Ott, “Capabilities and Reliability of LEDs and Laser Diodes” NASA Goddard Space Flight Center. 1997 (6) Hong, E., and N. Narendran. 2004. A method for projecting useful life of LED lighting systems. Third International Conference on Solid State Lighting, Proceeding of SPIE 5187: 93-99. (7) Narendran, N. and Y. Gu. 2005. Life of LED-based white light sources. IEEE/OSA Journal of Display Technology 1(1): 167-171. (8) Gu, Y., and N. Narendran. 2004. A non-contact method for determining junction temperature of phosphor-converted white LEDs. Third International Conference on Solid State Lighting, Proceedings of SPIE 5187: 107-114. (9) ASSIST Recommends. Published by the Lighting Research Center, Rensselaer Polytechnic Institute. Vol 1, issue 5. 2005

One or more of the following parameters may be measured or calculated and reported or used to control a light source. Further, one or more of these parameters may be tested per batch of light sources and/or batch of product and/or design. A parameter may be assumed to be a constant or updated as a function of measured parameters. The testing may be performed at standardized conditions (as in OEM testing) or under conditions that model actual use (for example, elevated temperature or expected overdriving electrical power). Power output can be approximated by assuming that for a given amount of time, the power output will decrease exponentially as a function of life (τ), with Pₒᵤₜ(t)=P₀e^{-t/τ}, where P₀=initial output, τ=exponential lifetime, t=time. The drive current's affect on a LED's life expectancy can be calculated by extrapolating the LEDs OEM rated drive current over a given amount of time: t₂=t₀(I₂/I⁰)⁻ⁿ, where t=time, I=drive current, n=empirical value; based on the LED system's sensitivity to increased current (1.5 to 2.0). The temperature affect on a LEDs life can be approximated as an exponential function of change in ambient temperature and the activation energy of the LED's semiconductor material, with t₂=t₀e^{Ea/k[1/T}0^{-1T}2^{]}, where t=time, Eₐ=semiconductor material activation energy (eV), T=temperature (K), k=Boltzman's Constant (1.38 E⁻²³ J/K). The temperature may be estimated, e.g., per session or per activation time, or measured with a temperature sensor. Accordingly, one or more of the formulae or derivative formulae may be used to process data in the device.

In one embodiment, a batch of light sources is tested at standard conditions and/or operating conditions (temperature, drive current, and/or test-to-failure in an end-user phototherapy device) to empirically measure one or more of the parameters, e.g., τ and/or power output as a function of cumulative light activation times and/or the empirical value of n. For example, a batch of LEDs is tested at a temperature and a drive current that reflects operation in the end-user device; the parameters (e.g., are entered into the processor(s) for a batch of end-user devices for use in control of the LEDs. In this manner, each end-user device is provided with an accurate set of parameters for control of the device. Accordingly, embodiments include providing a processor with light source parameter data as tested for the light source specifically used with the processor, with the data being standard conditions test data or test data gathered empirically for actual use conditions.

In another embodiment, test data at standard conditions (meaning the test conditions have not been altered to reflect actual use conditions, with OEM test data being a standard conditions test), is captured for each batch of light sources and the data is entered into the processors for a corresponding batch of end-user devices. The processor logic is adapted to account for use of standard as opposed to actual-use test data.

In another embodiment, a drive current for a light source is dynamically adjusted. A light source will degrade in a predictable manner for some devices. In such cases, the drive current to the light source can be increased to match the degradation. One option is to limit the amount of overdriving current that is available so that variation from device to device will not result in providing a light intensity that is in excess of the intended amount. One or more of the parameters may be monitored during actual use of the device and used to calculate a drive current for the light source that provides a predetermined light intensity. For example, a time of light source activation may be monitored and stored in the device and used to calculate a new drive current (an increased current) to drive the light source.

Some embodiments involve monitoring a plurality of parameters during actual use. For example, a time of actual use and an actual temperature may be monitored. The data may be used as described herein, e.g., to control the device to report degradation or lifetime status of a light source or to prevent light source activation.

In use, a processor in a unit may be programmed to account for one or more of these parameters and calculate its effect on light source life. When the life falls below a predetermined test value, the light source is deactivated and/or a message is provided indicating the same.

Embodiments also provide for the base and/or body to have user-interactive menus for selecting the desired therapy and head and suitable instructions, e.g., by way of a keypad and LCD display. For instance, a base or body may identify the type of head that is used and indicate the intended therapy, the session time, and/or instructions for use.

The unit may have a safety feature to prevent the light source from being directed to the user's eyes. This feature may be a distance detector (e.g., infrared) that turns off the LEDs when the distance from the head to a surface is too great. Alternatively, the feature measures capacitance of the skin and turns the device off when the head is not contacting the skin. Alternatively the device is a pressure-sensor that requires a minimum pressure at the face of the head. Kits or systems may be provided that include glasses that block light or filter out harmful wavelengths.

Various light sources and arrays of light sources can be configured with one or more LEDs, one or more laser diodes, one or more UVC bulbs, one or more lasers, one or more light bulbs, one or more ultraviolet light bulbs, one or more ultraviolet C light bulbs, or in any combination thereof. A wide variety of arrays have been proposed elsewhere. The following references are hereby incorporated by reference herein; in case of conflict, the instant specification controls: U.S. Pat. Nos. 7,267,673, 7,258,695, U.S. Pat. No. 7,201,765, U.S. Pat. No. 7,125,416, U.S. Pat. No. 7,198,633, U.S. Pat. No. 7,014,639, U.S. Pat. No. 6,918,922, U.S. Pat. No. 6,290,713, U.S. Pat. No. 6,221,095, U.S. Pat. No. 5,728,090, U.S. Pat. No. 5,549,660, US 2007/0167999, US 2006/009822, US 2006/0030908, US 20060064144, US 2005/0177093, US 2005/0065579, US 2005/0055070, US 2005/0228463, US 2004/0260365, and US 2003/016780.

Colors (wavelengths) and light sources may be used as per the intended use, e.g., red, blue, green, clear. An embodiment includes a plurality of light sources, with the device indicating (e.g., on the unit, the head, the base) when a subset of the sources is activated, or should be deactivated. By way of example, an embodiment is a unit that has a plurality of red LED and a plurality of blue LED lights together in a single replaceable head used to treat acne. The device indicates to the user which of the light sources are on: the red LEDs, the blue LEDs, or both. Another embodiment has a plurality of red LED and a plurality of blue LED lights together in a single replaceable head used to treat acne. The device indicates to the user which of the light sources are on: the red LEDs, the blue LEDs, or both. Another embodiment has a plurality of red LEDs and a plurality of blue LED lights together in a single replaceable head used to treat acne. The device indicates to the user which of the light sources are on: the red LEDs, the blue LEDs, or both. Alternatively, a laser diode may be included as an additional light source or as an alternative to one or more LEDs, e.g., a red laser diode instead of all of the red LEDs, or in addition to one or more LEDs. Alternatively, one or more laser diodes may be added to, or substituted for, one or more LEDs, e.g., a red LED may be a red laser diode, or a blue laser diode may be included.

An embodiment is a hand-held LED-based light therapy system. The device includes a base to receive a hand-held housing with a plurality of reversibly attachable heads that may be interchanged by the user. The heads have different LED lights. Users hold the housing and hold or move the head across their body tissue (e.g., skin or hair) to shine the LED light onto areas to receive the therapy. Pressing the device against the user can be a means to turn on the LEDs, or a hand operable switch may be used. The housing is put back in the base for recharging. The light is applied repeatedly over time to achieve the intended effect. Therapies include one or more of: acne (blue light), age Spots (Green Light), wrinkles (collagen production, with yellow and or red light), wound care (red and infra-red light), or a combination. A microprocessor in each head provides identifying information to the hand-held housing. A computer-readable medium in the head, the housing, or the base stores information about the actual use of the head, including the total time the LEDs in the head have been used. One aspect of this feature is that the LED intensity can be maintained at a helpful level because LEDs tend to lose intensity over time before they fail completely. At a predetermined time, a microprocessor notifies the user that the lifetime of the LEDs in the head has reached the end of its useful service life so that the user may obtain a new replacement head. The notification can be e.g., by sound, light, color bars, or so as provided herein. The notification can also provide a service life left indication. Optionally, the system will disable or otherwise refuse to operate the head when the predetermined service life is reached.

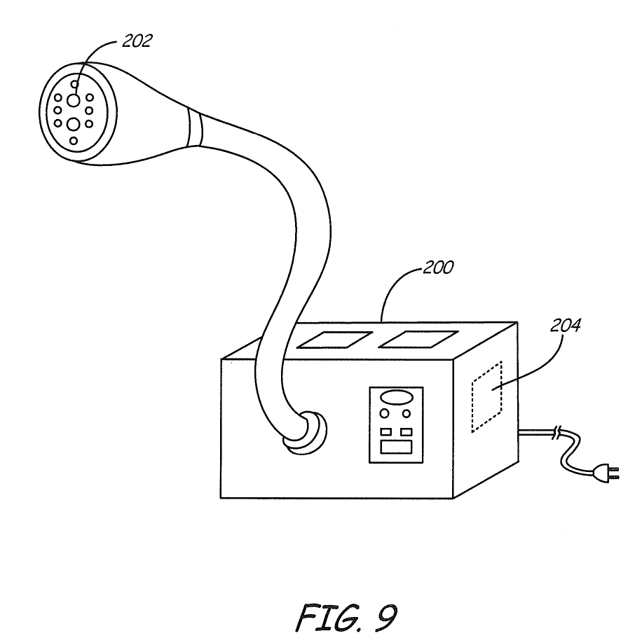

FIG. 9 is directed to a device wherein the light source is not designed to be part of a system of bodies with interchangeable heads. Instead, the light source is dedicated to the unit. The light source may be a permanent part of the unit (as in a device that is disposed of after the light lifetime is exhausted) or replaceable, or even reversibly removable, e.g., for cleaning or servicing. The body 200 has a light source 202, and a processor 204. The processor tracks usage of the light source prevents activation of the source when the light lifetime is exhausted and/or provides a message reflecting the status of light source lifetime. As is evident from the disclosure herein, there may be one or more processors that share the tracking and control functions, and computer-readable media may be used to store usage data and test data. Accordingly, and by way of example, the various devices incorporated herein by reference to other patent applications can be equipped with the usage-tracking features.

The phototherapy units or systems may be directed to a variety of light therapies. Examples include acne, wrinkles, hair loss, joint comfort or healing. For instance, phototherapy for acne may be accomplished with a blue light, e.g., about 50 microwatts per cm². Wound care may be provided with red light and/or infrared light at around 40 around 40-50 microwatts/cm2. Wrinkles may be treated with yellow light at about 50 microwatts/cm2. Green light also has uses for skin treatment, e.g., age spots, at, e.g, about 40 microwatts/cm2. Further details are provided in patent applications incorporated by reference herein elsewhere. The wavelengths and doses are provided for exemplary purposes and artisans will appreciate that a wide variety of therapies, doses, and light sources are contemplated. Two more examples are provided by way of illustration.

### Example 1

This example provides for a LED skin treatment device that applies therapeutic applications of light to skin, and uses different colors of light to treat many different skin conditions, e.g., age spots, wrinkles, acne and/or rosacea.

### Features

1. Replaceable LED head to facilitate using different colors for therapy, currently including red, blue, yellow, and green.
2. Monitor battery life and notify user when battery is low and needs charging.
3. Each LED head to monitor number of treatment cycles and notify user when head cycle count is getting close to the end.
4. Hand-held when applying light for a user
5. Ability to be use a universal charger.
6. Rechargeable Battery Pack.
7. Custom Charging Station Base

### Functional Options

1. Pressing Soft Touch On/Off button for predetermined time, e.g., more than 5 seconds initiates the treatment cycle and cause the LEDs to light.
2. During a treatment cycle, if the On/Off button is held for more than a predetermined time, e.g., 5 seconds, the device turns off. The LED display and indicators may also turn off.
3. When the treatment cycle completes, the indicators and LED display turn off.
4. The device may beep when it is turned on and/or when the treatment cycle is complete.
5. When the device is off, pressing the Soft Touch On/Off button for less than a predetermined time 1 second may cause the battery status and head life indicators to turn on.
6. Each replaceable head stores the number of cycles remaining. The maximum number of cycles available for a head will depend on the LED color used in the head. Alternative parameters may be measured, stored, or calculated as described herein.
7. Charging station may hold the device in an orientation that the battery life and head life indicators can be seen.
8. Three (blue) LED array on body may be used to show battery charge status. For instance, 1 lit to indicate needs charging, 2 lit to indicate a partial charge, and 3 lit to indicate a full charge.
9. Three (blue) LED array on body to indicate head life status. For instance, 1 lit to indicate head replacement is imminent, 2 lit to indicate approximately 50% of head life remains, 3 lit to indicate approximately 100% of head life remains. When the user tries to use a head with no more uses available, the bottom head life status indicator may flash and/or a beep will sound.
10. Two (2) red seven segment LED displays may be used to indicate to the user the number of minutes remaining in the treatment cycle.
11. Battery may be comprised of custom cells and have the ability to be replaced by the user.
12. May operate for 30 minutes without the need to recharge.
13. May operate via power cord when battery needs charging.
14. When the device is in the charging station, the battery status and head life indicators can be lit appropriately. When the device is recharging, the battery status lights can sequentially turn on.
15. Universal battery charger capable of charging at a 0.1 C rate.
16. Withstands daily use including, for instance: 5 treatment cycles per day/5 Years=9,125 cycles.

### Example 2

### Acne Therapy

This example provides for a design portable skin treatment device that treats acne and other skin conditions.

### Features:

1. Output Red laser and Blue LED light to treat acne.
2. Provide indication for when Red light is on or when Blue light is on.
3. Notify user when battery is low and needs charging.
4. Small and portable.
5. Ability to be sold globally using universal charger.
6. Head to be replaceable and to count number of uses.
7. Notify user when the head count number is getting close to the end.
8. Safety switch in the head to protect user from shining the laser in eyes.

### Functional Options

1. Give user an effective treatment in 6 minutes. The full treatment cycle will include 3 minutes of laser therapy and 3 minutes of blue light therapy.
2. To be able to operate for 30 minutes without the need to recharge.
3. Red LED used to indicate when the user needs to recharge the battery.
4. Connection method via connector on side of enclosure.
5. Light Source—1 red laser diode, 3 blue LEDs. Alternatively, red LEDs instead of, or in addition to, red laser diode.
6. Notify user which light is on: Bi-color LED (i.e. Red & Blue) to indicate when the laser is (Red) on and when the LEDs are (Blue) on.
7. Soft touch button to initiate cycle. Operational sequence: Press and hold the button for 5 seconds to turn on device. The device enabled for the full treatment cycle using both red laser and blue LEDs. Pressing the button again enables the red light only cycle. Pressing the button a second time enables the blue light only cycle.
8. After cycle is enabled, pressing the head against the area to be treated initiates the cycle. Moving the head away for the treatment area causes the cycle to stop.
9. When the device is on, pressing and holding the button for more than 5 seconds shuts off device. If device is on and no action occurs for more than 30 seconds, device automatically shuts off.
10. Head to maintain the count of the number of cycles for both the laser and blue LEDs. The laser shall stop functioning after 90 cycles. Likewise, the blue LEDs shall stop functioning after 90 cycles. Cycle count shall be decremented when the cycle is initiated.
11. Indicate to user when remaining cycles are getting low; e.g., the bicolor indicator LED may flash when the remaining cycles is less than 30.
12. Operate normally at ambient temperatures between 10° C.-30° C. and 15% to 80%. Relative humidity (Non-condensing).
13. A tone to signal the start, middle and end of the therapy cycle. A tone, along with flashing indicator LED, to indicate no more uses available for that color of light.
14. Battery charger capable of charging at a 0.1 C rate.

### Further Disclosure of Embodiments

1. A light therapy device with a light source and a processor that controls the light source in response to a usage history tracked by the device.
2. A light therapy system to activate a light source and apply therapeutic light to a tissue of a patient over a plurality of sessions comprising

a light source and a processor, wherein the processor stores usage data over a plurality of the sessions that comprise a duration of activation of the light source and tests the usage data to determine when degradation of the light source exceeds a predetermined test value.
3. A light therapy system to activate a light source and apply therapeutic light to a tissue of a patient over a plurality of sessions, with control of cumulative activation time of the light source, the system comprising: a head comprising a light source (e.g., a plurality of LEDs) that provides therapeutic light a body reversibly connectable to the head, with the body being electrically connected to the head to pass electrical power to the light source when the head is connected to the body; a computer-readable medium that stores light source usage (e.g., light activation time, driving amperage); a predetermined test value stored in a second computer-readable medium; and a processor programmed to make a usage comparison of the usage information and the predetermined test value, the processor allowing LED activation only when the usage comparison shows that a cumulative use of the head is exceeds the predetermined test value; wherein the body and the head, when connected, form a user-activatable, hand-held, light therapy unit for the user to administer the therapeutic light to the tissue of the user. Alternatively, with the processor providing a report (e.g., visual display, audio) instead or, or in addition to, allowing the a light activation to take place.
4. A light therapy unit to activate a light source and apply therapeutic light to a tissue of a patient over a plurality of sessions, with control of cumulative activation time of the light source, the system comprising: a light source (e.g., an LED or laser diode or bulb) that provides therapeutic light and is/are electrically activatable to emit light on the tissue; a first processor that captures light usage information and writes the usage information into a first computer-readable medium; a processor-readable cumulative use predetermined test value stored in a second computer-readable medium; and a second processor programmed to make a usage comparison of the usage information and the use predetermined test value, the processor allowing light source activation only when the usage comparison shows that a cumulative use of the light source is within a range established by the predetermined test value, the unit optionally being hand-held when in use.
5. A light therapy method involving using any or 1-4 or components thereof.
6. Any of 1-6 wherein the usage information is a time, a drive current, a temperature, or a number of sessions.
7. Any of 1-7 wherein the first processor and the second processor are one processor or more than two processors.
8. Any of 1-5 wherein the first computer-readable medium is located in the head and the second computer medium is located in the body.
9. Any of 1-9 wherein the first processor and the second processor are located in the body.
10. Any of 1-9 wherein one of the processors in the body updates the first computer-readable medium to store a cumulative usage.
11. Any of 1-10 wherein the LED usage information comprises a counter that is incremented or decremented by the first processor, the predetermined test value is a number of uses, and the comparison comprises comparing the counter to the predetermined test value.
12. Any of 1-11 wherein the LED usage information comprises a duration of LED activation and the first processor adds the duration of LED activation to a previously stored duration to generate a cumulative usage value in the first computer-readable medium.
13. Any of 1-12 wherein the first processor comprises a timer that measures the cumulative time of LED activation and stores the cumulative time in the first computer readable medium, with the cumulative use predetermined test value representing a maximum cumulative activation time.
14. Any of 1-13 wherein the body comprises a timer that captures a duration of the activation of the LEDs and the first processor writes the duration to the first computer readable medium.
15. Any of 1-14 wherein the first processor reads the usage information from the first computer-readable storage medium, adds the duration, and writes the resultant sum as the usage information on the first computer-readable storage medium.
16. Any of 1-15 wherein the first computer-readable storage medium comprises a computer memory or a register in a processor, and the second computer-readable storage medium comprises a computer memory or a register in a processor.
17. Any of 1-16 further comprising a session timer and an interval timer, with the session timer limiting the LED activation to a predetermined session duration, and the interval timer limiting the LED activation to a predetermined duration per interval.
18. Any of 1-17 wherein the LED comprises a red, blue, or white LED.
19. Any of 1-18 comprising a plurality of the heads, wherein each head comprises computer-readable storage medium for storage of cumulative usage data of the head.
20. Any of 1-19 wherein the first processor and the second processor are one processor or more than two processors.
21. Any of 1-20 wherein the first computer-readable medium is located in the head and the second computer medium is located in the body, the first processor and the second processor are located in the body, and one of the processors in the body updates the first computer-readable medium to store a cumulative usage.
22. Any of 1-21 wherein a current provided to the light source is dynamically adjusted to increase the current in response to a time of use or other parameter.
23. Any of 1-22 wherein a time of light source activation is monitored and stored in the device and used to calculate a new drive current (an increased current) to drive the light source.
24. Any of 1-23 wherein the head is deactivated by the unit if the degradation of the light source meets or exceeds about 5%, or alternatively, more than about 1%, 10%, or 20%.
25. Any of 1-24 including providing a processor with light source parameter data as tested for the light source specifically used with the processor, with the data being standard conditions test data or test data gathered empirically for actual use conditions.

A variety of embodiments of been described herein with certain features. The embodiments are by way of example and the features may be mixed-and-matched with each other as guided by the need to make an operable device and in accord with the spirit of this disclosure.

1. A light therapy system to activate a light source and apply therapeutic light to a tissue of a patient over a plurality of sessions comprising
a light source and a processor, wherein the processor is programmed to store usage data in a computer readable medium over a plurality of the sessions and to test the usage data to determine if degradation of the light source exceeds a predetermined test value.

2. The system of claim 1 wherein the processor program provides for test outcome to control the light source to be non-activatable when the degradation of the light source exceeds the predetermined test value.

3. The system of claim 1 wherein the test outcome provides a report comprising a visual or audio display and comprising a status of the light source degradation.

4. The system of claim 1 wherein the predetermined test value is a cumulative duration of activation.

5. The system of claim 1 wherein the usage data is a cumulative duration of light source activation that is updated by the processor in response to activation of the light source.

6. The system of claim 1 wherein the usage data further comprises light source drive current, light source temperature, or both drive current and temperature.

7. The system of claim 1 wherein the light source is located in a head reversibly connectable to a body that passes electrical power to the light source when the head and body are connected, with the head and body forming a hand-held unit.

8. The system of claim 7 wherein the data is stored in a computer-readable medium located in the head.

9. The system of claim 7 comprising a plurality of the heads that each comprise a computer readable medium to store the data for each head, with each head being reversibly connectable to the same body.

10. The system of claim 9 with one of the heads comprising a light source that provides a therapy to treat wrinkles.

11. The system of claim 9 with one of the heads comprising a light source that provides a therapy to treat acne.

12. The system of claim 11 wherein the processor is located in the body.

13. The system of claim 11 wherein the predetermined test value is stored in a computer-readable medium located in the body.

14. The system of claim 11 wherein the predetermined test value is stored in a computer-readable medium located in each of the heads.

15. The system of claim 14 wherein the predetermined test value is different for each of the heads.

16. The system of claim 1 wherein the processor is a plurality of processors that cooperate to measure the usage data, store the usage data, and test the usage data.

17. The system of claim 1 comprising a counter that is incremented or decremented by activation of the light source.

18. The system of claim 1 comprising a timer that measures a cumulative time of light source activation and stores the cumulative time as the usage data.

19. The system of claim 1 wherein the computer-readable storage medium comprises a computer memory or a register in a processor.

20. The system of claim 1 wherein the light source comprises a plurality of light emitting diodes (LEDs), a laser LED, or a combination thereof.

21. A light therapy system to activate a light source and apply therapeutic light to a tissue of a patient over a plurality of sessions, with control of cumulative usage of the light source, the system comprising:
a plurality of heads, with each at least one head comprising a plurality of light emitting diodes (LEDs) that provide therapeutic light and are electrically activatable to emit light on the tissue, wherein each head is non-identical and provides a distinct profile of therapeutic light;
a body reversibly connectable to each head, with the body being electrically connected to each head to pass electrical power to the LEDs when the head is connected to the body;
a computer-readable medium located in each head that stores LED usage data;
a computer-readable medium in the body that stores a cumulative, predetermined use test value; and
a processor in the body programmed to compare the usage data and the cumulative use test value, the processor allowing LED activation only when the comparison shows that a cumulative use of the head connected to the body exceeds a predetermined test value, with the processor further updating the usage data to reflect LED usage in the session;
wherein the body and any of the heads, when connected, form a user-activatable, hand-held, light therapy unit for the user to administer the therapeutic light to the tissue of the user.
